(19) United States
(12) Patent Application Publication
(10) Pub. No.: US 2024/0165033 A1
(43) Pub. Date: May 23, 2024

(54) SYSTEM OF MULTIPLE INTERLOCKING EMBEDMENT

(71) Applicant: CRAFT HEALTH PTE LTD, Singapore (SG)

(72) Inventors: Seng Han LIM, Singapore (SG); Wei Jiang GOH, Singapore (SG)

(21) Appl. No.: 18/554,502

(22) PCT Filed: May 6, 2022
(86) PCT No.: PCT/IB2022/054235

§ 371 Date: Oct. 9, 2023

Related U.S. Application Data
(60) Provisional application No. 63/184,934, filed on May 6, 2021.

(51) Int. Cl. A61K 9/20; A61J 3/10; A61K 9/00; B33Y 10/00; B33Y 30/00; B33Y 40/10; B33Y 40/20; B33Y 70/00; B33Y 80/00

(52) U.S. Cl. CPC A61K 9/2086; A61J 3/10; A61K 9/0053; A61K 9/2013; A61K 9/2027; A61K 9/2059; A61K 9/2095; B33Y 70/00; B33Y 80/00; B33Y 10/00; B33Y 30/00; B33Y 40/10; B33Y 40/20

(57) ABSTRACT

An oral dosage tablet for delivery of hydrophobic active ingredients to a patient and method of forming the same are described. The oral dosage tablet includes one or more layers of an active ingredient adjacent one or more layers of a lipid, such as a phospholipid. The layers are alternating and have a thickness of between 0.1 and 0.8 millimeters. The method of forming the tablet includes forming a lipid paste and an active ingredient paste of the respective components and applying alternating layers of each in layers of between 0.1 millimeters to 0.8 millimeters. The oral dosage tablet is subsequently dried and stored until treatment conmmences.

### CROSS-REFERENCE TO RELATED APPLICATIONS

This application claims the benefit of U.S. Provisional Application No. 63/184,934 filed May 6, 2021, the entire contents of which are hereby incorporated for all purposes in their entirety.

### BACKGROUND

Typically, orally administered drug dosages include liquids or powders administered as compressed tablets, coated tablets, or gelatin capsules containing a single dosage of a selected drug. Dosage regimens often require predetermined schedules of multiple dosages per day or over several days. Some active ingredients may not solubilize well in gastric fluid, resulting in poor absorption and poor bioavailability. In some typical systems, small molecules that may have demonstrated effectiveness and safety at the in vitro stage often fail at the in vivo stage due to less than favorable pharmacokinetics, typically due to absorption, distribution, metabolism, and elimination (ADME). The first hurdle, absorption, can be attributed to factors that are broadly classified as either solubility or permeability.

### BRIEF DESCRIPTION OF THE DRAWINGS

FIG. 1 depicts an example overview of a system for producing an oral dosage tablet with multiple interlocking layers, according to at least one example.

FIG. 2 depicts an example of an oral dosage tablet as the tablet is solubilized, according to at least one example.

FIG. 3 depicts an example testing apparatus used for testing of the oral dosage tablet described herein, according to at least one example.

FIG. 4 depicts an example schematic architecture for implementing techniques relating to generating instructions for manufacturing an oral dosage tablet, according to at least some embodiments.

FIG. 5 depicts an example of a computing device.

FIG. 6 depicts an example of a flowchart for a process of forming an oral dosage tablet for hydrophobic active ingredients, according to at least one example.

FIG. 7 depicts an example of an oral dosage tablet with multiple interlocking layers, according to at least one example.

### DETAILED DESCRIPTION

As described herein, an oral dosage tablet and method of manufacturing the same is provided to enable tablets using active ingredients that do not solubilize well in gastric or intestinal fluids, for example with hydrophobic active ingredients. In particular, due to the poor absorption, the active ingredients may be less effective than anticipated. Such active ingredients typically require a lipid and/or a phospholipid component to aid the solubilization of the active ingredients. The active ingredients may, for example, be classified according to the biopharmaceutics classification system (BCS) as class two or class four. Each of class two and class four exhibit low solubility, for example in gastric and/or intestinal fluids. Class four typically also exhibits reduced permeability as compared with class two active ingredients.

In some examples, small molecules that may have demonstrated effectiveness and safety at the in vitro stage may fail at the in vivo stage due to less than favorable pharmacokinetics, typically termed absorption, distribution, metabolism, and elimination (ADME). One major hurdle, absorption, can be attributed to factors that are broadly classified as either solubility or permeability. The oral dosage tablet described herein addresses the aspect of poor solubility or poor permeability, using a platform approach, where the active ingredient could be quickly formulated, and three dimensionally (3D) printed into a tablet.

As compared to typical systems, the oral dosage tablet described herein bypasses the need for a specific formulation stage for each active ingredient that may be used in a tablet, which requires careful selection of the oily phase and titration of both the phospholipids and/or surfactants and the hydrophobic drug (e.g., the active ingredient). In typical systems, the formulations stage is critical due to the need for a stable dispersion before the next formulation step of solidifying the formulation. The presence of the hydrophobic active ingredient, oil, and interaction with the aqueous environment adds complexity to the overall formulation. In comparison, the oral dosage tablet described herein simplifies the overall approach by keeping the oil and water phase physically separate and thereby only requiring formulation of the active ingredient, and not the active ingredient plus the lipids that are used to aid absorption. The oral dosage tablet described herein is also scalable, for size and production capacity, as tablets of varying sizes and shapes are manufactured in the same manner, and tablets used for trials, lab testing, or industrial manufacturing are each manufactured in a similar manner. The oral dosage tablet described herein also enables an ability to change active ingredients without changing the remainder of the tablet formulation. This contrasts with typical liposome or micelle production process, where new formulations must be developed for each active ingredient.

The oral dosage tablet described herein makes use of amphipathic molecules to improve the solubility of a poor soluble or hydrophobic active ingredient. This type of active ingredient is typically found in BCS classes 2 and 4. As used herein the term “amphipathic molecules” refers to a molecule composed of a hydrophilic portion, generally referred to as a “hydrophilic head”, and a hydrophobic portion generally referred to as a “hydrophobic tail”. Such molecules can form vesicular structures in which the hydrophobic tails associate to create a hydrophobic core and the hydrophilic heads associate with an external, aqueous environment. One such structure is a liposome in which a phospholipid bilayer is oriented in a generally circular/spherical configuration such that the hydrophilic heads of one layer surround an aqueous core while the hydrophilic heads of the other layer interact with a bulk aqueous phase. Another such structure is a micelle in which a monolayer arranges itself with hydrophobic tails associated in a hydrophobic core and hydrophilic heads interacting with a bulk aqueous phase. Also included are lipids and surfactants. The close proximity and increased contact surface area of the lipid and active ingredient layers improve the overall solubility of the active ingredient through the interaction of the amphipathic molecules with the active ingredient.

The oral dosage tablet described herein is 3D printed by applying multiple layers of active ingredients and phospholipids, typically in an alternating manner, to increase the surface area of the oily phase containing the active ingredient exposed to the aqueous environment (e.g., gastric fluid or intestinal fluid). As a result, the rate of solubilization of the hydrophobic active ingredient is improved over typical tablets. Additionally, doses of active ingredients may be personalized and adjusted through adjustments to the 3D printing, such as by printing thicker layers, thinner layers, more layers, or fewer layers, to adjust a dosage of the active ingredient without requiring a re-formulation and changes in production workflow to produce the tablet.

The oral dosage tablets described herein ensure that the thin layers of active ingredients are in close contact with a corresponding thin lipid layer. When the oral dosage tablet is orally ingested, the different layers start to disperse in the gastric environment. The increased surface area improves the ability for the lipid and/or phospholipids to solubilize the active ingredients. The increased surface area is achieved by 3D printing alternating thin layers of lipid and/or phospholipid and active ingredient.

Phospholipids typically consist of a hydrophilic head and hydrophobic end. This property is utilized in the oral dosage tablet where the hydrophobic active ingredient interacts with the hydrophobic end of the phospholipid, whereas the hydrophilic head of the phospholipid interacts with the aqueous environment. In some examples, the lipid and/or phospholipid may include any amphipathic components including surfactants, sphingolipids, ceramides, glycosphingolipids, and any combination thereof.

The phospholipids (or any of the compounds listed above) aid in the solubilization of hydrophobic active ingredients through the combination of micelles production, liposomes production or simply the cross interaction between the phospholipids and active ingredients. The net effect is the increased solubilization of the hydrophobic active ingredient. In contrast, lipids would dissolve the active ingredients which would otherwise remain as solid particles in the water-based gastric or intestinal fluids.

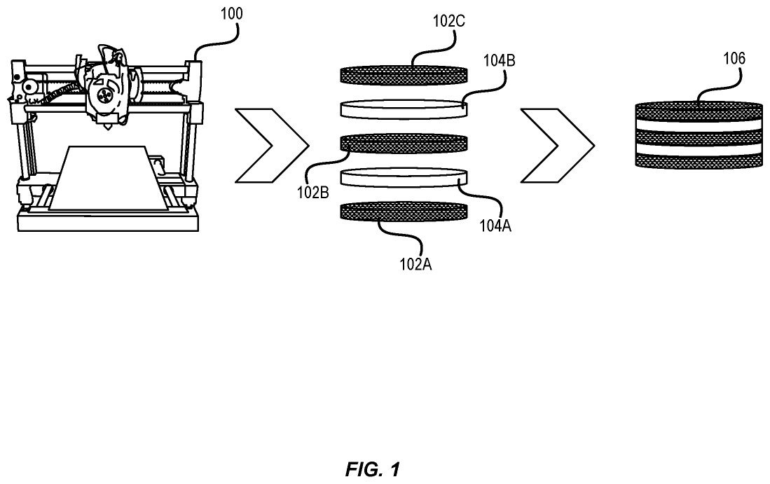

Turning now to the figures, FIG. 1 depicts an example overview of a system for producing an oral dosage tablet 106 with multiple interlocking layers, according to at least one example. In the example, the system includes a 3D printer 100 that is used to apply thin layers of a lipid and/or phospholipid and a hydrophobic or poorly soluble active ingredient. In some examples, the 3D printer may instead be replaced by any additive manufacturing system capably of applying subsequent alternating layers, for example through screen printing or other additive means.

The 3D printer 100 may have two printing heads, a single printing head, or multiple nozzles for printing the lipid and active ingredient layers. For example, a 3D printer 100 that includes printer nozzles for ejecting the lipid and the active ingredient layers may be able to produce oral dosage tablets 106 more efficiently than a 3D printer with a single supply that must alternate between a supply of lipid and a supply of active ingredient formulation.

The 3D printer 100 applies layers having a thickness of less than 1 millimeter of each of the lipid layer and the active ingredient layer. In some examples, the layers of each may be between 0.1 and 0.8 millimeters in thickness. In some examples, each layer may have a thickness of 0.6 millimeters each. In some examples, the layers of lipid and active ingredient may be applied in multiple passes of the 3D printer, for example, with each subsequent layering from the 3D printer 100 applying a thin (e.g. less than 0.2 mm) layer of the material and subsequently building up a layer of each to a desired thickness by applying multiple passes or layers via the 3D printer.

As shown in FIG. 1, the oral dosage tablet 106 is composed of several layers. In a particular example, a first layer 102A includes lipids and/or phospholipids. The first layer 102A may be applied on a substrate by the 3D printer 100 with a subsequent layer, second layer 104A applied on top of the first layer 102A. The second layer 104A may be the layer including the active ingredient. Additional alternating layers 102B, 104B, and 102C may be subsequently applied to produce the alternating layers of the oral dosage tablet 106. Though the oral dosage tablet 106 is shown having five layers, other numbers of layers may be used, greater than or less than 5 layers, such as three, four, six, seven, eight, nine, ten, or more layers.

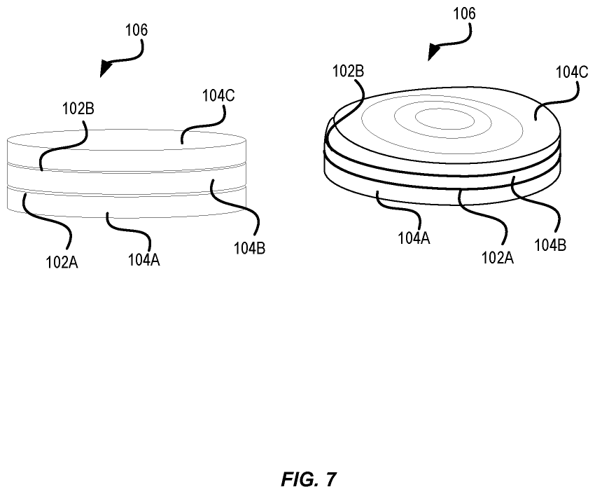

FIG. 7 depicts an example of an oral dosage tablet with multiple interlocking layers, according to at least one example. The oral dosage tablet is an example of the oral dosage tablet 106. In FIG. 7, the second layer 104A may be applied on a substrate by the 3D printer 100 with a subsequent layer, first layer 102A applied on top of the second layer 104A. The first layer 104A may be the layer including the active ingredient, such as abiraterone acetate (or any other targeted active ingredient for a drug), and the first layer 102A may be the layer that includes lipids and/or phospholipids. Additional alternating layers 104B, 102B, and 104C may be subsequently applied to produce the alternating layers of the oral dosage tablet 106.

Returning to FIG. 1, in some examples, the oral dosage tablet 106 may include more than two types of compounds in layers. For example, the oral dosage tablet 106 may include layer 104A having a first active ingredient while layer 104B includes a second, different, active ingredient from that contained in layer 104A. In some examples, the lipid and/or phospholipid layers may differ such that layers 102A, 102B, and 102C may contain the same formulation of lipids or may include multiple different formulations for different layers.

The layers of the oral dosage tablet 106 are applied by the 3D printer 100 as thin layers, less than one millimeter. In some examples, the layers may be applied through one or more passes of the 3D printer 100. The layers may have a thickness of between 0.1 millimeters to 0.8 millimeters in some examples. The thin layers, particularly of the active ingredient, increase the surface area of the active ingredient layers and enable dissolution and solubility rates for the active ingredients that exceed typical oral dosage tablets. In some examples, the layers including the lipids may have a different thickness than the layers having the active ingredients. For example, the active ingredient layers may be thicker than the lipid layers or the active ingredient layers may be thinner than the lipid layers.

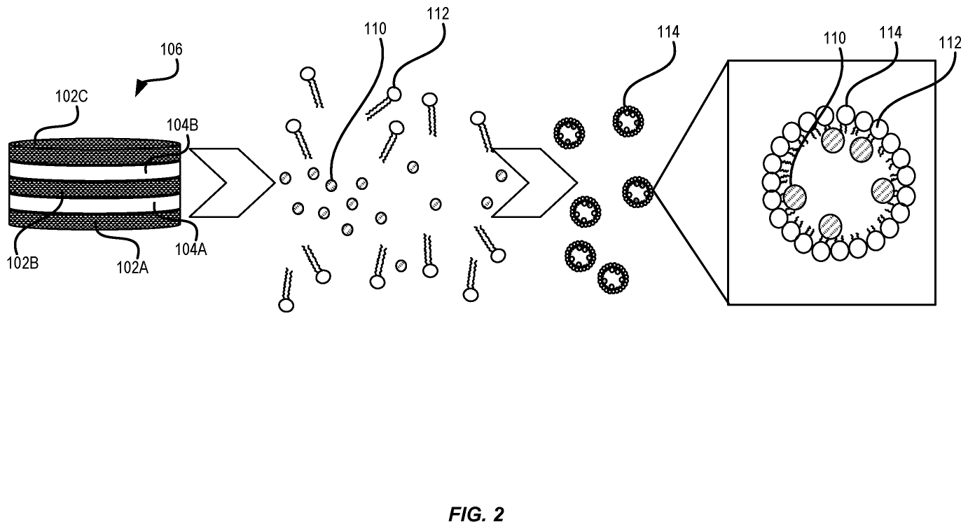

FIG. 2 depicts an example of an oral dosage tablet 106 as the oral dosage tablet 106 is solubilized, according to at least one example. Due to the close proximity of the lipid layers 102 to the active ingredient layers 104, the hydrophobic active ingredient 110 of the active ingredient layer 104 is quickly solubilized through the interaction of the lipids 112 with the hydrophobic active ingredient 110, through a combination of micellar formation, liposomal formation or the interaction of the phospholipids with the hydrophobic active ingredient. FIG. 2 highlights the formation of a micelle 114, encapsulating the hydrophobic active ingredient 110 in its interior while the perimeter is formed of the lipids 112.

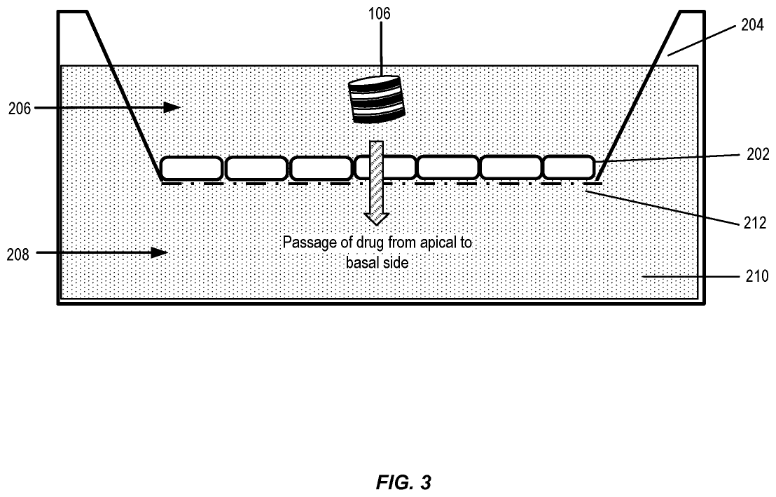

FIG. 3 depicts an example testing apparatus used for testing of the oral dosage tablet 106 described herein, according to at least one example. As shown in FIG. 3, the efficacy of the oral dosage tablet 106 correlates to the ability of the active ingredient to be effectively solubilized and absorbed across the gastrointestinal tract. The testing apparatus may mimic the operation of the oral dosage tablet 106 within a patient and therefore illustrates the oral dosage tablet 106 in practice. The apparatus of FIG. 3 includes a typical Caco-2 permeability assay, where Caco-2 cells 202 are grown as a monolayer in a transwell insert 204. The apparatus includes a chamber 206 where the oral dosage tablet 106 is introduced, as the oral dosage tablet 106 would be introduced upon ingestion by a patient. The apparatus also includes a basal chamber 208 containing a medium 210 separated from the chamber 206 by a semi-permeable membrane that mimics, in combination with the Caco-2 cells 202, the lining the stomach or gastrointestinal tract. The oral dosage tablet 106 is then added to the transwell insert 204 and the amount of active ingredient that passes through the Caco-2 cells 202 is then assayed from the other end of the transwell insert 204 using standard quantitative methods of either high performance liquid chromatography (HPLC) or liquid chromatography mass spectroscopy (LCMS) techniques. The amount of active ingredient that passes through the Caco-2 cells 202 is significantly higher using the oral dosage tablet 106 than the active ingredient alone.

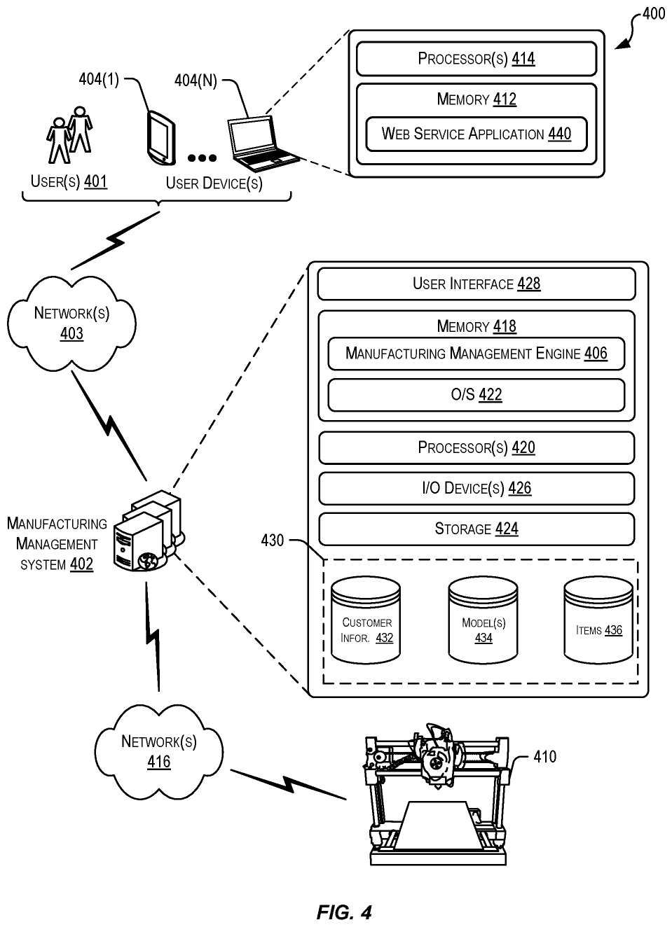

FIG. 4 depicts an example schematic architecture 400 for implementing techniques relating to generating instructions for manufacturing an oral dosage tablet as described herein, according to at least some embodiments. The architecture 400 may include a manufacturing management system 402 in communication with one or more user devices 404(1)-404(N) (hereinafter, “the user device 404”) via one or more networks 403 (hereinafter, “the network 403”). The manufacturing management system 402 communicates with a user device 404 and a production apparatus 410. Using any suitable software, application, etc. running on the user device 404 or otherwise, a user 401 may provide input to the manufacturing management system 402 to design an oral dosage tablet (e.g., oral dosage tablet 106 in FIGS. 1-3). The design of the oral dosage tablet 106 may include the size, shape, number of layers, thickness of layers, types of lipids, types of active ingredients, and other such design factors described herein. The user device 404 may be operable by one or more users 401 (hereinafter, “the user 401”) to interact with the manufacturing management system 402. The network 403 may include any one or a combination of many different types of networks, such as cable networks, the Internet, wireless networks, cellular networks, and other private and/or public networks. The user 401 may be any suitable user including, for example, customers of an electronic marketplace that are associated with the manufacturing management system 402, or any other suitable user.

The production apparatus 410 may include any suitable additive and/or subtractive manufacturing apparatus configured to perform any suitable manufacturing process. For example, the production apparatus 410 is illustrated as an extrusion deposition type of apparatus such as a 3D printer. Other suitable manufacturing apparatuses may be configured to perform processes including, for example, a screen printing machine, a digital ink jet printing machine, a flexo printing machine, a ultra violet (UV) lithography printing machine, laser printing machine, a pad printing machine, a laminated object manufacturing machine, a stereolithography machine, and/or any other suitable additive and/or subtractive production machine. Additional methods and apparatuses for manufacturing may be used in some examples including vacuum forming, thermoplastic forming, casting, injection molding, molding, and the like.

The architecture 400 may also include the production apparatus 410 in communication with at least the manufacturing management system 402 via a secondary network 416. The secondary network 416 may include any one or a combination of many different types of networks as described elsewhere herein.

Turning now to the details of the user device 404, the user device 404 may be any suitable type of computing device such as, but not limited to, a tablet, a mobile phone, a smart phone, a personal digital assistant (PDA), a laptop computer, a desktop computer, a cloud computing device, or any other suitable device capable of communicating with the manufacturing management system 402 via the network 403 or any other suitable network. For example, the user device 404(1) is illustrated as an example of a smart phone, while the user device 404(N) is illustrated as an example of a laptop computer.

The user device 404 may include a web service application 440 within memory 412. Within the memory 412 of the user device 404 may be stored program instructions that are loadable and executable on processor(s) 414, as well as data generated during the execution of these programs. Depending on the configuration and type of user device 404, the memory 412 may be volatile (such as random access memory (RAM)) and/or non-volatile (such as read-only memory (ROM), flash memory, etc.). The web service application 440, stored in the memory 412, may allow the user 401 to interact with the manufacturing management system 402 via the network 403. Such interactions may include, for example, interacting with user interfaces provided by the manufacturing management system 402, selecting oral dosage tablet 106 designs, customizing oral dosage tablet 106 capsule designs (e.g., by adjusting a size, thickness, or components within each of the layers), and placing orders for oral dosage tablets 106, performing any other interaction described herein or relating to obtaining tablets, and any other suitable client-server interactions. The manufacturing management system 402, whether associated with the electronic marketplace or not, may host the web service application 440.

The manufacturing management system 402 may include one or more service provider computers, and may host web service applications. These servers may be configured to host a website (or combination of websites) viewable on the user device 404 (e.g., via the web service application 440). The user 401 may access the website to view items (e.g., capsules) that can be ordered from the manufacturing management system 402 (or an electronic marketplace associated with the manufacturing management system 402). These may be presentable to the user 401 via the web service applications.

The manufacturing management system 402 may include at least one memory 418 and one or more processing units (or processor(s)) 420. The processor 420 may be implemented as appropriate in hardware, computer-executable instructions, software, firmware, or combinations thereof. Computer-executable instruction, software, or firmware implementations of the processor 420 may include computer-executable or machine-executable instructions written in any suitable programming language to perform the various functions described. The memory 418 may include more than one memory and may be distributed throughout the manufacturing management system 402. The memory 418 may store program instructions that are loadable and executable on the processor(s) 420, as well as data generated during the execution of these programs. Depending on the configuration and type of memory including the manufacturing management system 402, the memory 418 may be volatile (such as random access memory (RAM)) and/or non-volatile (such as read-only memory (ROM), flash memory, or other memory). The memory 418 may include an operating system 422 and one or more application programs, modules, or services for implementing the techniques described herein including at least a manufacturing management engine 406. In some examples, the production apparatus 410 is configured to perform the techniques described herein with reference to the manufacturing management system 402, including the manufacturing management engine 406.

The manufacturing management system 402 may also include additional storage 424, which may be removable storage and/or non-removable storage including, but not limited to, magnetic storage, optical disks, and/or tape storage as well as private or public cloud networks. The disk drives and their associated computer-readable media may provide non-volatile storage of computer-readable instructions, data structures, program modules, and other data for the computing devices. The additional storage 424, both removable and non-removable, are examples of computer-readable storage media. For example, computer-readable storage media may include volatile or non-volatile, removable or non-removable media implemented in any suitable method or technology for storage of information such as computer-readable instructions, data structures, program modules, or other data. As used herein, modules, engines, and components, may refer to programming modules executed by computing systems (e.g., processors) that are part of the manufacturing management system 402, the user device 404, and/or the production apparatus 410.

The manufacturing management system 402 may also include input/output (I/O) device(s) and/or ports 426, such as for enabling connection with a keyboard, a mouse, a pen, a voice input device, a touch input device, a display, speakers, a printer, or other I/O device.

The manufacturing management system 402 may also include a user interface 428. The user interface 428 may be utilized by an operator or one of the users 401 to access portions of the manufacturing management system 402. In some examples, the user interface 428 may include a graphical user interface, web-based applications, programmatic interfaces such as application programming interfaces (APIs), or other user interface configurations. The manufacturing management system 402 may also include a data store 430. In some examples, the data store 430 may include one or more data stores, databases, data structures, or the like for storing and/or retaining information associated with the manufacturing management system 402. Thus, the data store 430 may include databases, such as a customer information database 432, a model database 434, and a content item database 436.

The customer information database 432 may be used to retain information pertaining to customers of the manufacturing management system 402, such as the user 401. Such information may include, for example, customer account information (e.g., electronic profiles for individual users), demographic information for customers, payment instrument information for customers (e.g., credit card, debit cards, bank account information, and other similar payment processing instruments), account preferences for customers, shipping preferences for customers, purchase history of customers, oral dosage tablet models, customer material preferences, and other similar information pertaining to a particular customer and sets of customers of the manufacturing management system 402. In some examples, the customer information may be encrypted and decrypted when needed using typical encryption techniques. In some examples, the customer information may be de-identified or anonymized and instead merely present generic profiles that can be selected from for manufacturing. In some examples, the information retained in the customer information database 432 may be shared with and/or received from the electronic marketplace.

The model database 434 may be used to store three-dimensional models or designs of oral dosage tablets 106. The model database 434 may be referenced when the manufacturing management engine 406 attempts to identify a particular three-dimensional item or a particular oral dosage tablet design, or generate manufacturing instructions for a particular tablet. The model database 434 may be configured to store any suitable data in any suitable format (e.g., computer-aided drafting (CAD) file such as a STereoLithography file or .STL format) capable of storing a representation of a three-dimensional item.

The digital content item database 436 may be used to retain information about digital content items for which oral dosage tablet designs are available. For example, the digital content item database 436 may include a table that includes all digital content items available for purchase in the electronic marketplace, information about the design of the different oral dosage tablets such as the active ingredients included and the dosage.

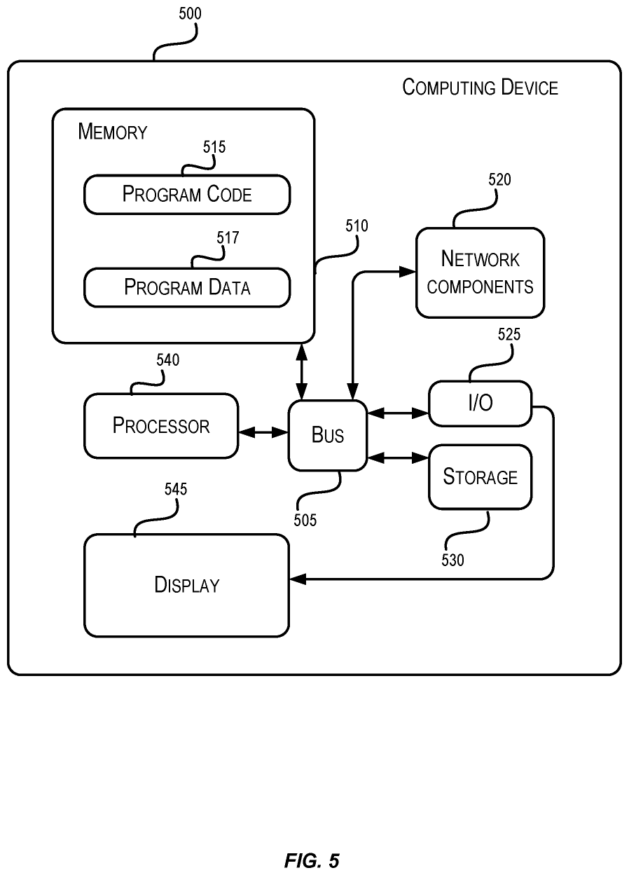

Any suitable computing system or group of computing systems can be used for performing the operations or methods described herein. FIG. 5 depicts an example of a computing device 500. In an embodiment, a computing device, such as user device 404 or manufacturing management system 402 combines the one or more operations and data stores depicted as separate subsystems herein.

FIG. 5 illustrates a block diagram of an example of a computing device 500. Computing device 500 can be any of the described computers herein including, for example, user device 404 or manufacturing management system 402. The computing device 500 can be or include, for example, an integrated computer, a laptop computer, desktop computer, tablet, server, or other electronic device.

The computing device 500 can include a processor 540 interfaced with other hardware via a bus 505. A memory 510, which can include any suitable tangible (and non-transitory) computer readable medium, such as RAM, ROM, EEPROM, or the like, can embody program components (e.g., program code 515) that configure operation of the computing device 500. Memory 510 can store the program code 515, program data 517, or both. In some examples, the computing device 500 can include input/output (“I/O”) interface components 525 (e.g., for interfacing with a display 545, keyboard, mouse, and the like) and additional storage 530.

The computing device 500 executes program code 515 that configures the processor 540 to perform one or more of the operations described herein. The program code 515 may be resident in the memory 510 or any suitable computer-readable medium and may be executed by the processor 540 or any other suitable processor.

The computing device 500 may generate or receive program data 517 by virtue of executing the program code 515. For example, oral dosage tablet designs, drug characteristics, formulations, and patient treatment profiles are all examples of program data 517 that may be used by the computing device 500 during execution of the program code 515.

The computing device 500 can include network components 520. Network components 520 can represent one or more of any components that facilitate a network connection. In some examples, the network components 520 can facilitate a wireless connection and include wireless interfaces such as IEEE 802.11, Bluetooth, or radio interfaces for accessing cellular telephone networks (e.g., a transceiver/antenna for accessing CDMA, GSM, UMTS, or other mobile communications network). In other examples, the network components 520 can be wired and can include interfaces such as Ethernet, USB, or IEEE 1394.

Although FIG. 5 depicts one computing device 500 with a single processor 540, the system can include any number of computing devices 500 and any number of processors 540. For example, multiple computing devices 500 or multiple processors 540 can be distributed over a wired or wireless network (e.g., a Wide Area Network, Local Area Network, or the Internet). The multiple computing devices 500 or multiple processors 540 can perform any of the steps of the present disclosure individually or in coordination with one another.

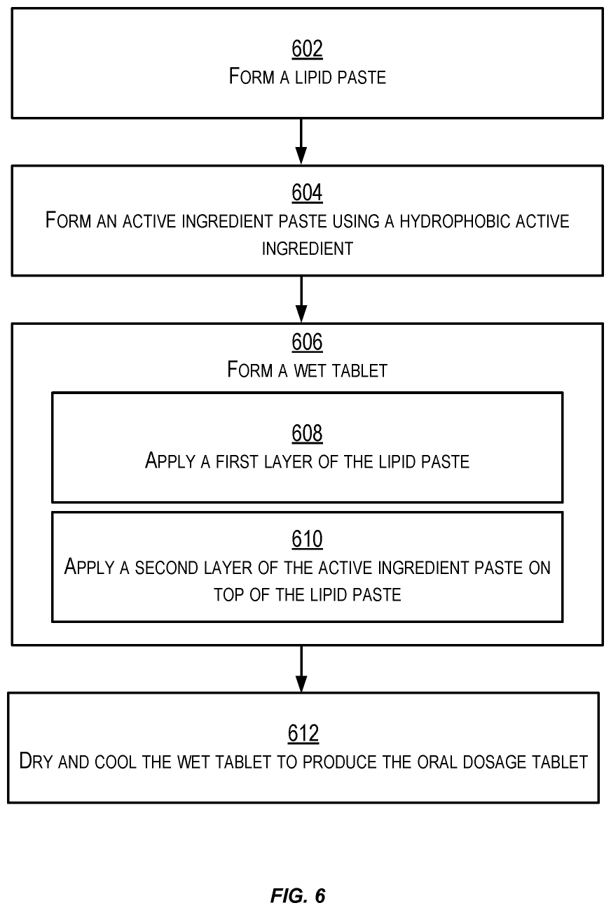

FIG. 6 depicts an example of a flowchart for a process of forming an oral dosage tablet for hydrophobic active ingredients, according to at least one example. The oral dosage tablet, such as the oral dosage tablet 106 in FIGS. 1-3 may be produced by a method as described herein, including one or more steps and systems for applying or printing layers of active ingredients and lipids. In some examples, the method for forming the oral dosage tablet 106 includes operation 602 of forming a lipid or phospholipid paste. The lipids and/or phospholipids would be pre-selected and formulated. The formulation of the lipid and/or phospholipid layer could be used with many different active ingredients. The lipid layer is formulated to contain lipids such as medium chain triglycerides, phospholipids such as lecithin and a small proportion of surfactant such as poloxamer and are mixed to obtain a uniform paste that is 3D printable. A suitable solvent or increase in temperature to the transition temperature of the main lipid constituent may be included to facilitate the process of mixing and 3D printing.

The method for forming the oral dosage tablet 106 may also include operation 604 of forming an active ingredient paste. The formulation for each active ingredient layer may be fixed once determined. The paste for the active ingredient layer may be formed primarily through the mixing of hydrophilic polymers such as Polyvinylpyrrolidone with a water-based co-solvent. The active ingredient layer is formulated to contain the required active ingredient, hydrophilic binders such as polyvinylpyrrolidone (PVP) and a disintegrant such as sodium starch glycolate and are mixed to obtain a uniform paste. As the main change in formulation is the active ingredient, formulation changes can be made quickly by only changing the active ingredient layer and therefore, accelerating time to market for different oral dosage tablets 106.

The method for forming the oral dosage tablet 106 may also include operation 606 of forming a wet tablet. Forming the wet tablet can involve operations 608, where a first layer of a lipid paste is applied, and operation 610, where a second layer of the active ingredient paste is applied on top of the lipid paste. The thin layers of the lipid paste and the active ingredient paste may be applied through the use of a 3D printer, screen printer, or other application method that applies thin layers of a pate or paste-like material to a substrate. The layers may be applied at a thickness of between 0.3 to 0.8 millimeters. In some examples, prior to applying the thin layers, the lipid and/or the active ingredient paste may be degassed and loaded into a syringe or other dispensing device. The degassing may be performed using a centrifuge. Degassing the paste ensures that the layers will be applied consistently and evenly without air bubbles or pockets.

A traditional tablet machine could potentially be used to produce oral dosage tablet 106. In such examples, a layer of lipid would be compacted onto a layer of pre-compacted tablet. This may present challenges due to the sticky and soft nature of lipids, making it difficult for a tablet to be compacted in this manner. Furthermore, repeated layering within a single layer would take additional amount of time for traditional tablet machine, as compared to produce a single layer tablet.

The method for forming oral dosage tablet 106 may also include operation 612 of drying and cooling the oral dosage tablets after applying the thin layers. The drying of the oral dosage tablet 106 removes any excess water or moisture from the layers that may have been added to formulate the paste for the lipid or active ingredient layers. The cooling adds rigidity to the lipids and hardens the tablet. Once dried and stored in a low temperature environment, the tablets become more stable and durable for storage and transportation.

The method for forming the oral dosage tablet 106 may also include storing the oral dosage tablets 106 in airtight, light proof packaging at a particular temperature to prevent the lipid layers of the oral dosage tablet 106 from becoming rancid. The oral dosage tablets 106 may be stored at or below four degrees Celsius in airtight containers to prevent the lipid layers from becoming rancid. In some examples, the containers may also block light, such as ultraviolet light, from contacting the oral dosage tablet 106. The oral dosage tablet 106 thereby becomes stable and enables storage for extended periods of time without losses in efficacy of the tablet.

While the present subject matter has been described in detail with respect to specific aspects thereof, it will be appreciated that those skilled in the art, upon attaining an understanding of the foregoing, may readily produce alterations to, variations of, and equivalents to such aspects. Numerous specific details are set forth herein to provide a thorough understanding of the claimed subject matter. However, those skilled in the art will understand that the claimed subject matter may be practiced without these specific details. In other instances, methods, apparatuses, or systems that would be known by one of ordinary skill have not been described in detail so as not to obscure claimed subject matter. Accordingly, the present disclosure has been presented for purposes of example rather than limitation, and does not preclude the inclusion of such modifications, variations, and/or additions to the present subject matter as would be readily apparent to one of ordinary skill in the art.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included herein are for ease of explanation only and are not meant to be limiting.

Aspects of the methods disclosed herein may be performed in the operation of such computing devices. The system or systems discussed herein are not limited to any particular hardware architecture or configuration. A computing device can include any suitable arrangement of components that provide a result conditioned on one or more inputs. Suitable computing devices include multi-purpose microprocessor-based computer systems accessing stored software that programs or configures the computing system from a general purpose computing apparatus to a specialized computing apparatus implementing one or more aspects of the present subject matter. Any suitable programming, scripting, or other type of language or combinations of languages may be used to implement the teachings contained herein in software to be used in programming or configuring a computing device. The order of the blocks presented in the examples above can be varied—for example, blocks can be re-ordered, combined, and/or broken into sub-blocks. Certain blocks or processes can be performed in parallel.

1. A method of forming an oral dosage tablet for hydrophobic active ingredients, the method comprising:
forming a lipid paste;
forming an active ingredient paste using a hydrophobic active ingredient;
forming a wet tablet by:
applying a first layer of the lipid paste; and
applying a second layer of the active ingredient paste on top of the lipid paste, wherein each of the first and second layer are between 0.1 to 0.8 mm in thickness; and drying and cooling the wet tablet to produce the oral dosage tablet.

2. The method of claim 1, wherein the lipid paste comprises a lipid, a phospholipid, or other amphipathic components, and combinations thereof.

3. The method of claim 1, wherein forming the lipid paste comprises heating a lipid to a transition temperature of the lipid.

4. The method of claim 1, wherein forming the lipid paste comprises adding a solvent to a lipid.

5. The method of claim 1, wherein forming the active ingredient paste comprises mixing the singular or multiple active ingredients with a binder and a disintegrant.

6. The method of claim 5, wherein the binder comprises polyvinylpyrrolidone and/or other similar excipients with similar functions to the binder.

7. The method of claim 5, wherein the disintegrant comprises sodium starch glycolate and/or other similar excipients with similar functions to the disintegrant.

8. The method of claim 1, further comprising degassing the active ingredient paste and the lipid paste before forming the wet tablet.

9. The method of claim 1, wherein the first layer and the second layer are sequentially deposited using a three dimensional (3D) printer.

10. An oral dosage tablet for hydrophobic active ingredients, comprising:
a plurality of lipid layers, at least one lipid layer of the plurality of lipid layers comprising a lipid or phospholipid and having a first thickness of between 0.1 to 0.8 mm; and
a plurality of active ingredient layers, at least one active ingredient layer of the plurality of active ingredient layers comprising a hydrophobic active ingredient and having a second thickness of between 0.1 to 0.8 mm, wherein the plurality of lipid layers and the plurality of active ingredient layers alternate such that each lipid layer is in contact with a respective active ingredient layer.

11. The oral dosage tablet of claim 10, wherein the at least one active ingredient layer comprises a disintegrant.

12. The oral dosage tablet of claim 11, wherein the disintegrant comprises sodium starch glycolate and or other similar excipients with similar functions to the disintegrant.

13. The oral dosage tablet of claim 10, wherein the at least one active ingredient layer comprises a binder.

14. The oral dosage tablet of claim 13, wherein the binder comprises polyvinylpyrrolidone and or other similar excipients with similar functions to the binder.

15. The oral dosage tablet of claim 10, wherein each of the plurality of lipid layers and each of the plurality of active ingredient layers has the first thickness and the second thickness of between 0.1 to 0.6 mm.

16. A system for producing an oral dosage tablet, comprising:
a lipid paste forming device that forms a lipid paste comprising a phospholipid;
an active ingredient paste forming device that forms a paste comprising a hydrophobic active ingredient by combining the hydrophobic active ingredient with a binder and a solvent;
a paste layering device to apply alternating layers of the lipid paste and the active ingredient paste onto a substrate, wherein each layer is applied at a thickness of between 0.1 to 0.8 mm; and
a drying and cooling system for drying and cooling a wet tablet produced by the paste layering device.

17. The system of claim 16, further comprising a degassing device for removing air bubbles from the lipid paste and the active ingredient paste before application by the paste layering device.

18. The system of claim 16, wherein the paste layering device comprises a three dimensional (3D) printer, wherein the 3D printer applies the lipid paste and the active ingredient paste in alternating layers onto the substrate in a tablet shape.

19. (canceled)

20. The system of claim 16, wherein the lipid paste forming device heats the phospholipid to a transition temperature to form the lipid paste.

21. The system of claim 16, wherein the system comprises a three dimensional (3D) printer, wherein the oral dosage tablet is 3D printed by applying multiple layers of hydrophobic active ingredients and phospholipids to increase a surface area of an oily phase containing a hydrophobic active ingredient exposed to an aqueous environment, thereby improving a rate of solubilization of the hydrophobic active ingredient.
